(19) United States
(12) Patent Application Publication
(10) Pub. No.: US 2026/0026878 A1
(43) Pub. Date: Jan. 29, 2026

(54) High Density Catheter

(71) Applicant: ST. JUDE MEDICAL, CARDIOLOGY DIVISION, INC., Saint Paul, MN (US)

(72) Inventors: Bruce EBNER, Shorewood, MN (US); Vlad POPOV, Plymouth, MN (US); Hong CAO, Maple Grove, MN (US)

(73) Assignee: ST. JUDE MEDICAL, CARDIOLOGY DIVISION, INC., Saint Paul, MN (US)

(21) Appl. No.: 18/840,669

(22) PCT Filed: Feb. 22, 2023
(86) PCT No.: PCT/US2023/013632

§ 371 Date: Aug. 22, 2024

Related U.S. Application Data
(60) Provisional application No. 63/313,152, filed on Feb. 23, 2022.

(51) Int. Cl. A61B 18/14; A61B 18/00; A61M 25/01

(52) U.S. Cl. CPC A61B 18/1492; A61B 2018/00267; A61B 2018/00577; A61B 2018/00875; A61B 2018/1467; A61M 25/0127

(57) ABSTRACT

An electrode assembly for a catheter includes a central spline assembly. a first frame assembly. and a second frame assembly. The central spline assembly includes a first electrode portion that includes first electrodes. and a central spline end portion. The central spline assembly includes a spline frame support aperture. Each of the first and second frame assemblies include an understructure member that extends through the spline frame support aperture.

### CROSS REFERENCE TO RELATED APPLICATION DATA

The present application claims the benefit of U.S. Provisional Application No. 63/313,152 filed Feb. 23, 2022; the full disclosure which is incorporated herein by reference in its entirety for all purposes.

### BACKGROUND

Electrophysiological (EP) catheters can be configured for use in diagnosing and/or treating cardiac arrythmias. A cardiac arrythmia may be manifest in one or more observable medical conditions including, for example, an irregular heart rate, loss of synchronous atrioventricular contractions, and inadequate flow of blood through a chamber of the heart, which can lead to a variety of symptomatic and/or asymptomatic ailments and even death. Electrical activity of a patient's heart can be measured and assessed to determine whether the patient's heart exhibits a pathological electrical condition(s) associated with the occurrence of the cardiac arrythmia. Following diagnosis of the pathological electrical condition(s), a suitable treatment(s) can be used to selectively alter the patient's heart tissue to reduce or eliminate the pathological electrical condition to reduce or eliminate occurrence of the cardiac arrythmia. The treatment can include, for example, radio frequency (RF) ablation, pulsed field ablation, cryoablation, laser ablation, chemical ablation, high-intensity focused ultrasound ablation, microwave ablation, and/or other ablation treatments.

### BRIEF SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Where the term “planar” or, similarly, “plane” or “coplanar” is used herein, it should be understood to refer to a topological plane. In other words, a “plane” may not be “flat” in a Cartesian coordinate system, but rather represents a two-dimensional distribution that is planar in a topological sense. Likewise, where the term “linear” is used herein, it should be understood to refer to a topological plane. In other words, a “linear” may not be “straight line” in a Cartesian coordinate system, but rather represents a one-dimensional distribution that is linear in a topological sense.

High-density catheters described herein include a distal electrode assembly that includes a planar two-dimensional array of electrodes. In many embodiments, the distal electrode assembly includes a central flexible spline assembly, a first flexible spline frame assembly, and a second flexible spline frame assembly. The central flexible spline assembly includes a first electrode support section that supports a first linear sequence of the planar two-dimensional array of electrodes. The first flexible spline frame assembly includes a second electrode support section and a third electrode support section. The second electrode support section supports a second linear sequence of the planar two-dimensional array of electrodes. The third electrode support section supports a third linear sequence of the planar two-dimensional array of electrodes. The second flexible spline frame assembly includes a fourth electrode support section and a fifth electrode support section. The fourth electrode support section supports a fourth linear sequence of the planar two-dimensional array of electrodes. The fifth electrode support section supports a fifth linear sequence of the planar two-dimensional array of electrodes. The first, second, third, fourth, and fifth electrode support sections are spaced apart so that the planar two-dimensional array of electrodes have a planar distribution. In many embodiments, the first flexible spline frame assembly and the second flexible spline frame assembly share a symmetrical configuration and have opposite orientations in the distal electrode assembly that differ by 180 degrees. The central flexible spline assembly is centrally located in the distal electrode assembly. The central flexible spline assembly includes a spline frame support aperture. Each of the first flexible spline frame assembly and the second flexible spline frame assembly extend through the spline frame support aperture so that the position of each of the first flexible spline frame assembly and the second flexible spline frame assembly is restrained relative to the central flexible spline assembly. The spline frame support aperture is configured to accommodate relative sliding of end portions of the first and second flexible spline frame assemblies through the spline frame support aperture so as to reduce stresses induced in the distal electrode assembly by collapsing the distal electrode assembly from an expanded configuration to a collapsed configuration in which the distal electrode assembly is advanced through a patient's vasculature into the patient's heart for deployment in the expanded configuration.

Thus, in one aspect, a catheter includes an elongated catheter shaft and an electrode assembly. The electrode assembly includes a proximal connector, a central spline assembly, a first frame assembly, and a second frame assembly. The proximal connector is attached to a distal end of the elongated catheter shaft. The central spline assembly includes a first proximal portion, a first electrode portion, a first spline distal portion, and first spline electrodes. The first proximal portion is attached to and extends distally from the proximal connector. The first electrode portion extends distally from the first proximal portion. The first spline distal portion extends distally from a distal end of the first electrode portion and comprises a spline frame support aperture. The first spline electrodes are distributed along the first electrode portion. The first frame assembly includes a second proximal portion, a second electrode portion, a third proximal portion, a third electrode portion, a first frame end portion, second spline electrodes, and third spline electrodes. The second proximal portion is attached to and extends distally from the proximal connector on a first side of the central spline assembly. The second electrode portion extends distally from the second proximal portion. The third proximal portion is attached to and extends distally from the proximal connector on a second side of the central spline assembly opposite to the first side of the central spline assembly. The third electrode portion extends distally from the third proximal portion. The first frame end portion extends between a distal end of each of the second electrode portion and the third electrode portion and extends through the spline frame support aperture. The second spline electrodes are distributed along the second electrode portion. The third spline electrodes distributed along the third electrode portion. The second frame assembly includes a fourth proximal portion, a fourth electrode portion, a fifth proximal portion, a fifth electrode portion, a second frame end portion, fourth spline electrodes, and fifth spline electrodes. The fourth proximal portion is attached to and extends distally from the proximal connector on the first side of the central spline assembly. The fourth electrode portion extends distally from the fourth proximal portion. The fifth proximal portion is attached to and extends distally from the proximal connector on the second side of the central spline assembly. The fifth electrode portion extends distally from the fifth proximal portion. The second frame end portion extends between to a distal end of each of the fourth electrode portion and the fifth electrode portion and extends through the spline frame support aperture. The fourth spline electrodes are distributed along the fourth electrode portion. The fifth spline electrodes are distributed along the fifth electrode portion. In many embodiments, the first frame end portion crosses the second frame end portion within the spline frame support aperture.

In many embodiments, the electrode assembly has a planar deployed configuration. For example, in many embodiments, the first electrode portion, the second electrode portion, the third electrode portion, the fourth electrode portion, and the fifth electrode portion are coplanar in an expanded configuration of the electrode assembly. In many embodiments, the third electrode portion is disposed between the first electrode portion and the fifth electrode portion in the expanded configuration of the electrode assembly and the fourth electrode portion is disposed between the first electrode portion and the second electrode portion in the expanded configuration of the electrode assembly.

In many embodiments, the electrode assembly is configured to self-expand to a deployed configuration. For example, in many embodiments, the electrode assembly is configured to self-expand from a collapsed configuration within a lumen of a guiding sheath to an expanded configuration of the electrode assembly via retraction of the guiding sheath relative to the electrode assembly or advancement of the electrode assembly relative to the guiding sheath. In many embodiments, the first frame end portion extends distally from the distal end of each of the second electrode portion and the third electrode portion to a distal most point of the first frame end portion. In many embodiments, the distal most point of the first frame end portion remains on the first side of the central spline assembly and moves distally relative to the central spline assembly during reconfiguration of the electrode assembly from the collapsed configuration to the expanded configuration. In many embodiments, the second frame end portion extends distally from the distal end of each of the fourth electrode portion and the fifth electrode portion to a distal most point of the second frame end portion. In many embodiments, the distal most point of the second frame end portion remains on the second side of the central spline assembly and moves distally relative to the central spline assembly during reconfiguration of the electrode assembly from the collapsed configuration to the expanded configuration. In many embodiments, a respective segment of each of the first frame end portion and the second frame end portion moves through the spline frame support aperture during reconfiguration of the electrode assembly from the collapsed configuration to the expanded configuration.

In many embodiments, a single configuration is used for both the first frame assembly and the second frame assembly. In such embodiments, the orientation of the second frame assembly in the electrode assembly can be rotated 180 degrees relative to the orientation of the first frame assembly in the electrode assembly.

In many embodiments, the electrode assembly includes one or more locations sensors (e.g., one or more magnetic location sensors). For example, the central spline assembly can include one or more central spline location sensors. The one or more central spline magnetic location sensors can be disposed at any suitable location(s) in the central spline assembly including, but not limited to, distal to the first spline electrodes, in between any of the first spline electrodes, and/or proximal to the first spline electrodes. In some embodiments, the first frame assembly includes one or more first frame location sensors. The one or more first frame location sensors can be disposed at any suitable location(s) within the first frame assembly including, but not limited to, distal to the second spline electrodes, distal to the third spline electrodes, in between any two of the second spline electrodes, in between any two of the third spline electrodes, proximal to the second spline electrodes, and/or proximal to the third spline electrodes. In some embodiments, the second frame assembly includes one or more second frame location sensors. The one or more second frame location sensors can be disposed at any suitable location(s) within the second frame assembly including, but not limited to, distal to the fourth spline electrodes, distal to the fifth spline electrodes, in between any two of the fourth spline electrodes, in between any two of the fifth spline electrodes, proximal to the fourth spline electrodes, and/or proximal to the fifth spline electrodes.

In many embodiments, the electrode assembly is configured to conform to a tissue to place the first spline electrodes, the second spline electrodes, the third spline electrodes, the fourth spline electrodes, and the fifth spline electrodes in contact with the tissue.

The electrodes can have any suitable spacing. For example, in some embodiments, a center-to-center distance between adjacent electrodes of the first spline electrodes, the second spline electrodes, the third spline electrodes, the fourth spline electrodes, and the fifth spline electrodes is between 2 and 10 millimeters.

Any suitable number and/or arrangement of electrodes can be used. For example, in some embodiments, each of the first spline electrodes, the second spline electrodes, the third spline electrodes, the fourth spline electrodes, and the fifth spline electrodes include at least 4 electrodes. In some embodiments, the first electrode portion, the second electrode portion, the third electrode portion, the fourth electrode portion, and the fifth electrode portion are of equal length.

The catheter can further include any suitable conventional components. For example, in some embodiments, the catheter includes a fluid delivery lumen fluidly coupled to a source of an irrigation fluid and configured to deliver the irrigation fluid to the electrode assembly.

The catheter can be configured for any suitable medical task. For example, in some embodiments, the electrode assembly is configured for ablation therapy.

In another aspect, a catheter system can include a catheter configured as described above and controller circuitry communicatively coupled to the first spline electrodes, the second spline electrodes, the third spline electrodes, the fourth spline electrodes, and the fifth spline electrodes. The controller circuitry can be configured to sample electrical signals received from the first spline electrodes, the second spline electrodes, the third spline electrodes, the fourth spline electrodes, and the fifth spline electrodes. In some embodiments, the electrophysiological system further includes a display communicatively coupled to the controller circuitry and the controller circuitry is configured to generate and display an electrophysiology map indicative of one or more electrical characteristics of tissue contacted by the first spline electrodes, the second spline electrodes, the third spline electrodes, the fourth spline electrodes, and the fifth spline electrodes.

For a fuller understanding of the nature and advantages of the present invention, reference should be made to the ensuing detailed description and accompanying drawings.

### BRIEF DESCRIPTION OF THE DRAWINGS

FIG. 1 and FIG. 2 are isometric views of a distal electrode assembly of a catheter, in accordance with various embodiments of the present disclosure.

FIG. 3 is a top view of the electrode assembly of FIG. 1.

FIG. 4 is a side view of the electrode assembly of FIG. 1 conformed to a tissue to interface electrodes of the electrode assembly with the tissue.

FIG. 5 is a top view of the electrode assembly of FIG. 1 in a collapsed configuration.

FIG. 6 is an end cross-sectional view of the electrode assembly of FIG. 1 in the collapsed configuration shown in FIG. 5.

FIG. 7 is an end cross-sectional view of the electrode assembly of FIG. 1 in an expanded configuration.

FIG. 8 is a top view of understructure of the electrode assembly of FIG. 1.

FIG. 9 is an isometric view of the understructure shown in FIG. 8.

FIG. 10 shows a cross-sectional view of the understructure shown in FIG. 9.

FIG. 11 illustrates an example medical device localization system that can be employed in conjunction with the electrode assembly of FIG. 1.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

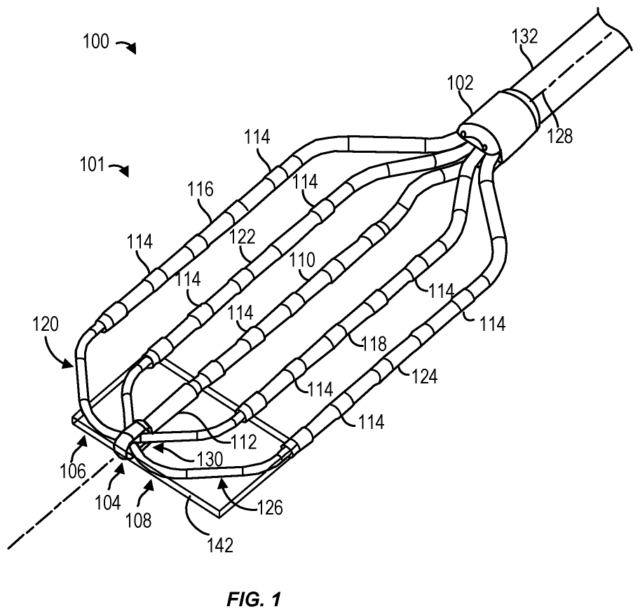

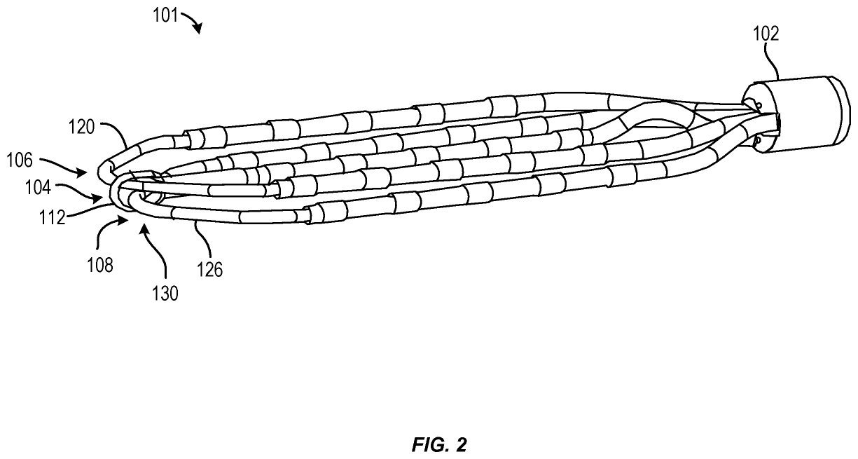

Referring now to the drawings wherein like reference numerals are used to identify identical components in the various views, FIG. 1 and FIG. 2 are isometric views of a distal electrode assembly 101 of a high-density catheter 100, in accordance with various embodiments of the present disclosure. The distal electrode assembly 101 includes a connector 102, a central flexible spline assembly 104, a first flexible spline frame assembly 106, and a second flexible spline frame assembly 108. The central flexible spline assembly 104 includes a first proximal portion 109, a first electrode portion 110, and a distal portion 112. The first proximal portion 109 of the central flexible spline assembly 104 is attached to and extends distally from the connector 102. The first electrode portion 110 extends distally from the first proximal portion 109. The first electrode portion 110 includes a first linear sequence of a planar two-dimensional array of electrodes 114 of the distal electrode assembly 101. The first flexible spline frame assembly 106 includes a second proximal portion 115, a second electrode portion 116, and a first flexible spline frame end portion 120, a third electrode portion 118, and a third proximal portion 119. The second proximal portion 115 of the first flexible spline frame assembly 106 is attached to and extends distally from the connector 102. The second electrode portion 116 extends distally from the second proximal portion 115. The second electrode portion 116 includes a second linear sequence of the planar two-dimensional array of electrodes 114 of the distal electrode assembly 101. The third proximal portion 119 of the first flexible spline frame assembly 106 is attached to and extends distally from the connector 102. The third electrode portion 118 extends distally from the third proximal portion 119. The third electrode portion 118 includes a third linear sequence of the planar two-dimensional array of electrodes 114 of the distal electrode assembly 101. The second flexible spline frame assembly 108 includes a fourth proximal portion 121, a fourth electrode portion 122, and a second flexible spline frame end portion 126, a fifth electrode portion 124, and a fifth proximal portion 125. The fourth proximal portion 121 of the second flexible spline frame assembly 108 is attached to and extends distally from the connector 102. The fourth electrode portion 122 extends distally from the fourth proximal portion 121. The fourth electrode portion 122 includes a fourth linear sequence of the planar two-dimensional array of electrodes 114 of the distal electrode assembly 101. The fifth proximal portion 125 of the second flexible spline frame assembly 108 is attached to and extends distally from the connector 102. The fifth electrode portion 124 extends distally from the fifth proximal portion 125. The fifth electrode portion 124 includes a fifth linear sequence of the planar two-dimensional array of electrodes 114 of the distal electrode assembly 101.

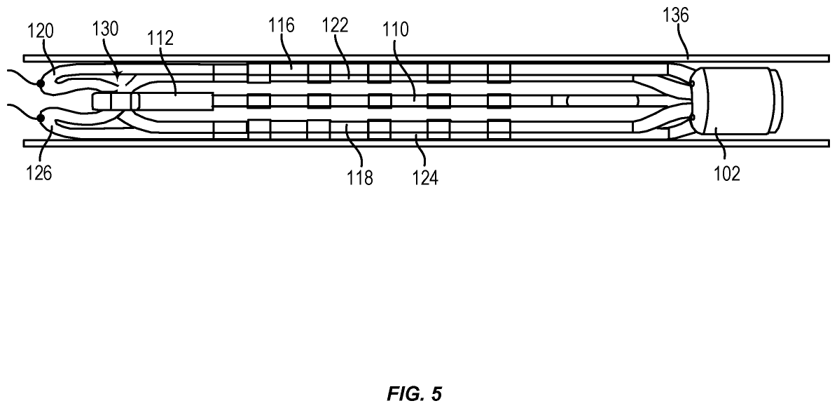

The first electrode portion 110 of the central flexible spline assembly 104 is aligned with a central longitudinal axis 128 of the high-density catheter 100. The first electrode portion 110 has a straight-line configuration in an undeformed state (e.g., when not being flexed to conform to a tissue surface). The distal portion 112 of the central flexible spline assembly 104 extends distally from the distal end of the first electrode portion 110. The distal portion 112 forms a spline frame support aperture 130. Each of the first flexible spline frame end portion 120 and the second flexible spline frame end portion 126 extends through the spline frame support aperture 130 and can move through the spline frame support aperture 130 during reconfiguration of the distal electrode assembly 101, such as, for example, during flexing of the distal electrode assembly 101 to conform the distal electrode assembly 101 with a tissue surface to interface the electrodes 114 with the tissue surface and during reconfiguration of the distal electrode assembly 101 between a collapsed configuration (shown in FIG. 5) and an expanded configuration (shown in FIG. 1). In the expanded configuration shown in FIG. 1, each of the second electrode portion 116, the third electrode portion 118, the fourth electrode portion 122, and the fifth electrode portion 124 is offset from and parallel to the first electrode portion 110. The distal electrode assembly 101, however, can be configured so that each of the second electrode portion 116, the third electrode portion 118, the fourth electrode portion 122, and the fifth electrode portion 124 is offset from and oriented non-parallel to the first electrode portion 110. Moreover, although the electrodes 114 are distributed in a regular two-dimensional grid pattern in the illustrated embodiment, the electrodes 114 can be distributed in any suitable arrangement in which the electrodes 114 have suitable offsets from each other including, but not limited to, non-orthogonal distribution patterns in which the electrodes on adjacent electrode portions 110, 116, 118, 122, 124 are offset longitudinally along the axis 128.

In the illustrated embodiment, a single configuration is used for both the first flexible spline frame assembly 106 and the second flexible spline frame assembly 108. The first flexible spline frame assembly 106 is attached to the connector 102 in a first orientation relative to the connector 102. The second flexible spline frame assembly 108 is attached to the connector 102 in a second orientation that is 180 degrees rotated around the central longitudinal axis 128 relative to the first orientation of the first flexible spline frame assembly 106. Additionally, the first flexible spline frame assembly 106 is offset to a first side of the axis 128 so that the third electrode portion 118 is disposed between the first electrode portion 110 and the fifth electrode portion 124. The second flexible spline frame assembly 108 is offset to a second side of the axis 128 (opposite to the first side of the axis 128) so that the fourth electrode portion 122 is disposed between the second electrode portion 116 and the first electrode portion 110. The first flexible spline frame end portion 120 is shaped so as to pass through the spline frame support aperture 130 on a first side of the plane in which the electrode portions 110, 116, 118, 122, 124 are disposed (e.g., above the plane with respect to the view direction of FIG. 1). Due to the similar configuration and the 180 degree opposite orientation of the second flexible spline frame assembly 108 relative to the first flexible spline frame assembly 106, the second flexible spline frame end portion 126 passes through the spline frame support aperture 130 on a second side of the plane in which the electrode portions 110, 116, 118, 122, 124 are disposed (e.g., below the plane with respect to the view direction of FIG. 1). Therefore, the first flexible spline frame end portion 120 and the second flexible spline frame end portion 126 cross each other within the spline frame support aperture 130 with the first flexible spline frame end portion 120 passing over the second flexible spline frame end portion 126 relative to the view direction of FIG. 2. In some embodiments, however, the second flexible spline frame end portion 126 passes over the first flexible spline frame end portion 120 within the spline frame support aperture 130 relative to the view direction of FIG. 2.

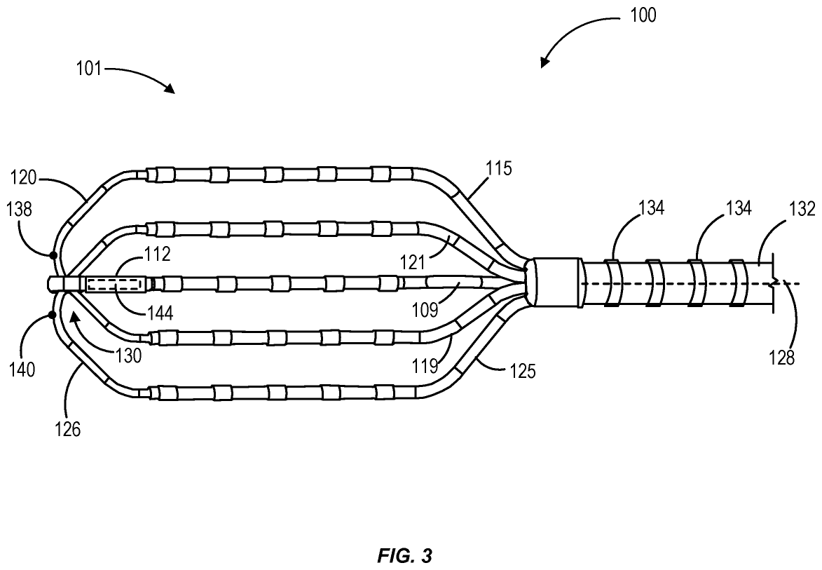

FIG. 3 is a top view of the electrode assembly 101. Each of the central flexible spline assembly 104, the first flexible spline frame assembly 106, and the second flexible spline frame assembly 108 is configured to have a suitable flexural flexibility so that the distal electrode assembly 101 can be conformed to a target tissue surface for any suitable medical purpose such as, but not limited to, to measure electrical activity of the heart via the electrodes 114, to perform a medical treatment using the electrodes 114, and/or to generate a surface model of the target tissue surface. In the illustrated embodiment, each of the first electrode portion 110, the second electrode portion 116, the third electrode portion 118, the fourth electrode portion 122, and the fifth electrode portion 124 has five of the electrodes 114, but can have any suitable alternate number of the electrodes 114 (e.g., 1, 2, 3, 4, 6, 7, 8, or more). The electrodes 114 are spaced apart on the distal electrode assembly 101 to form a two-dimensional distribution of the electrodes 114.

The electrodes 114 can be configured for use in diagnostic, therapeutic, and/or mapping procedures. For example and without limitation, the electrodes 114 can be configured for use in electrophysiological studies, pacing, cardiac mapping, and/or ablation. In some embodiments, the electrodes 114 can be configured for use in performing unipolar or bipolar ablation, which can be used to create specific lines or patterns of lesions. In some embodiments, the electrodes 114 can receive electrical signals from the heart, which can be used for electrophysiological studies. In some embodiments, the electrodes 114 can perform a location or position sensing function related to cardiac mapping.

In many embodiments, the high-density catheter 100 includes a catheter shaft 132. The catheter shaft 132 includes a proximal end and a distal end. The distal end of the catheter shaft 132 can be attached to the connector 102. The catheter shaft 132 can be made to have a suitable flexural compliance so that it can be advanced through suitable paths through a patient's vasculature. In some embodiments, the catheter shaft 132 includes one or more ring electrodes 134 disposed along a length of the catheter shaft 132. The ring electrodes 134 can be configured for use in diagnostic, therapeutic, and/or mapping procedures.

The high-density catheter 100 can further include any additional suitable components. For example, the high-density catheter 100 can further include other conventional components such as, for example and without limitation, a temperature sensor, additional sensors or electrodes, ablation elements (e.g., ablation tip electrodes for delivering RF ablative energy, high intensity focused ultrasound ablation elements, etc.), and corresponding conductors or leads.

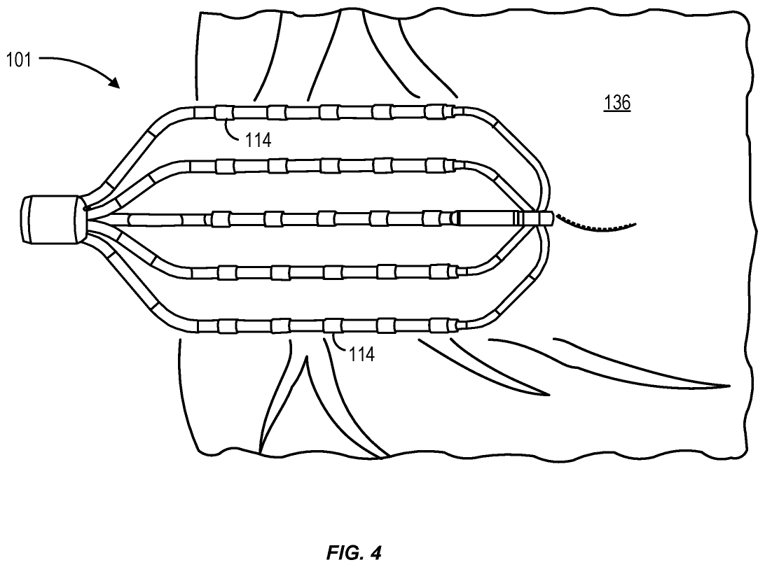

As depicted in FIG. 4, the distal electrode assembly 101 is configured to be conformable to a tissue 136 (e.g., cardiac tissue) to interface the electrodes 114 with the tissue 136. In many embodiments, the distal electrode assembly 101 has a suitable flexibility to accommodate suitable flexure of the electrode assembly 101 in response to suitable interface forces between the distal electrode assembly 101 and the tissue 136.

The configuration of the distal electrode assembly 101 facilitates insertion of the electrode assembly 101 into a delivery catheter or introducer, deployment of the distal electrode assembly 101 within the heart, and withdrawal of the distal electrode assembly 101 from the patient through the deliver catheter or introducer by accommodating relative movement between the central flexible spline assembly 104, the first flexible spline frame assembly 104, and the second flexible spline frame assembly 108, which can serve to avoid inducing high localized strains in the distal electrode assembly 101 that may result absent the relative movement accommodation.

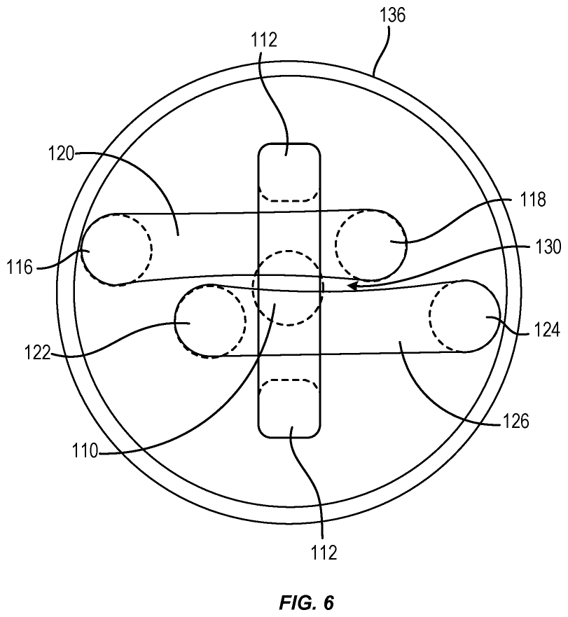

FIG. 5 is a top view of the distal electrode assembly 101 in a collapsed configuration within a lumen of an introducer 136. FIG. 6 is an end cross-sectional view of the distal electrode assembly 101 in the collapsed configuration. In the collapsed configuration, each of the second electrode portion 116, the third electrode portion 118, the fourth electrode portion 122, and the fifth electrode portion 124 are disposed adjacent to the first electrode portion 110 within the lumen of the introducer 136. During reconfiguration of the distal electrode assembly 101 from the expanded configuration (shown in FIG. 1) to the collapsed configuration, each of the first flexible spline frame assembly 106 and the second flexible spline frame assembly 108 is collapsed transverse to the central longitudinal axis 128 to fit within the lumen of the introducer 136. During the collapsing of the first flexible spline frame assembly 106, the first flexible spline frame end portion 120 is deformed from the expanded shape shown in FIG. 1 to collapsed shape shown in FIG. 5. During the deformation of the first flexible spline frame end portion 120 from the expanded shape to the collapsed shape, a distal most point 138 of the first flexible spline frame end portion 120 moves distally relative to the distal portion 112 of the central flexible spline assembly 104 and a segment of the first flexible spline frame end portion 120 passes through the spline frame support aperture 130 from the side of the distal portion 112 on which the third electrode portion 118 is disposed to the side of the distal portion 112 on which the second electrode portion 116 is disposed. By accommodating the sliding of the segment of the first flexible spline frame end portion 120 through the spline frame support aperture 130 during the collapsing of the first flexible spline frame assembly 106, the magnitudes of strains within the first flexible spline frame assembly 106 and the central spline assembly 104 in the collapsed configuration may be reduced substantially relative to a configuration in which the first flexible spline frame assembly 106 and the central spline assembly 104 are fixedly attached to a shared distal end member. Likewise, during the collapsing of the second flexible spline frame assembly 108, the second flexible spline frame end portion 126 is deformed from the expanded shape shown in FIG. 1 to collapsed shape shown in FIG. 5. During the deformation of the second flexible spline frame end portion 126 from the expanded shape to the collapsed shape, a distal most point 140 of the second flexible spline frame end portion 126 moves distally relative to the distal portion 112 of the central flexible spline assembly 104 and a segment of the second flexible spline frame end portion 126 passes through the spline frame support aperture 130 from the side of the distal portion 112 on which the fourth electrode portion 122 is disposed to the side of the distal portion 112 on which the fifth electrode portion 124 is disposed. By accommodating the sliding of the segment of the second flexible spline frame end portion 126 through the spline frame support aperture 130 during the collapsing of the second flexible spline frame assembly 108, the magnitudes of strains within the second flexible spline frame assembly 108 and the central flexible spline assembly 104 in the collapsed configuration may be reduced substantially relative to a configuration in which the second flexible spline frame assembly 108 and the central flexible spline assembly 104 are fixedly attached to a shared distal end member. Reduced strains within the distal electrode assembly 101 in the collapsed configuration serve to increase the operational life of the distal electrode assembly 101.

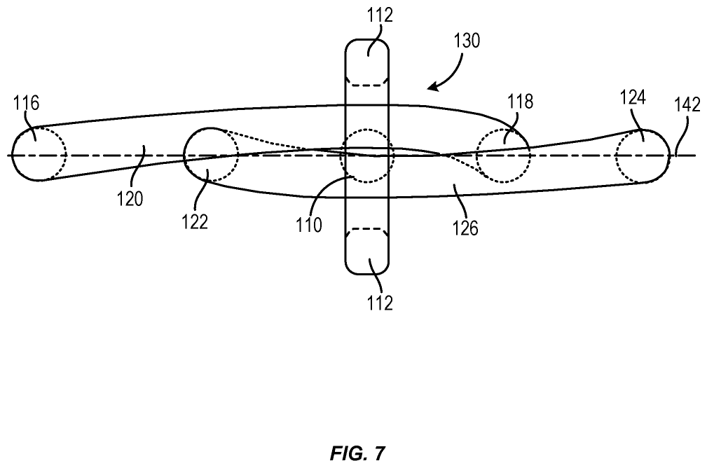

FIG. 7 is an end cross-sectional view of the distal electrode assembly 101 in the expanded configuration shown in FIG. 1. In the illustrated expanded configuration, each of the first electrode portion 110, the second electrode portion 116, the third electrode portion 118, the fourth electrode portion 122, and the fifth electrode portion 124 are disposed in a common plane 142. The first flexible spline frame end portion 120 and the second flexible spline frame end portion 126 are shaped to cross within the spline frame support aperture 130. Additionally, the first flexible spline frame end portion 120 is shaped to attach to and extend between the distal end of the second electrode portion 116 and the distal end of the third electrode portion 118, which are disposed in the common plane 142. Likewise, the second flexible spline frame end portion 126 is shaped to attach to and extend between the distal end of the fourth electrode portion 122 and the distal end of the fifth electrode portion 124. As described herein the first flexible spline frame assembly 106 and the second flexible spline frame assembly 108 can share a common shape and have 180 degrees opposite orientations in the distal electrode assembly 101 relative to the central longitudinal axis 128.

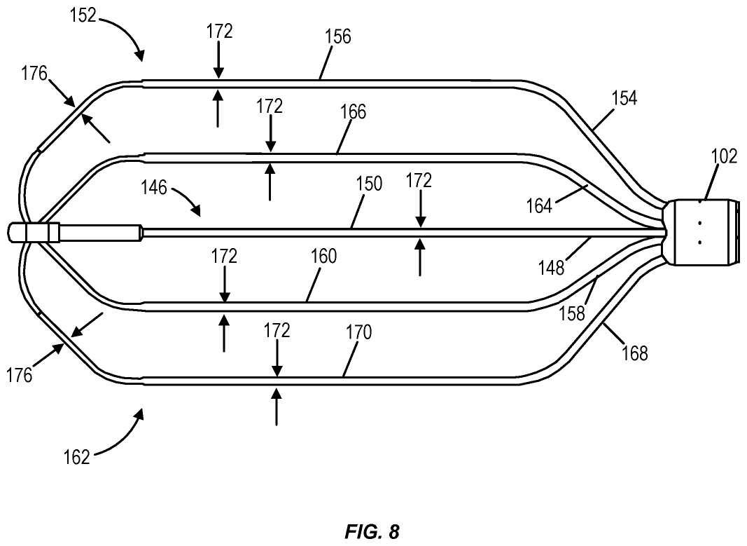

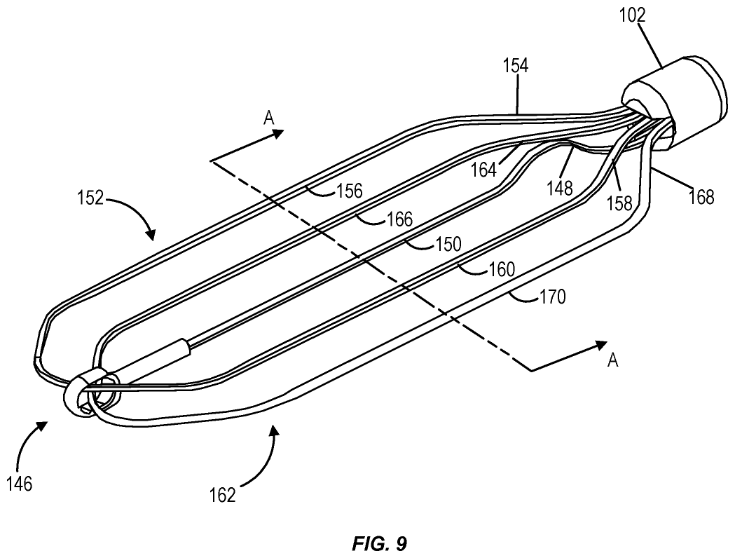

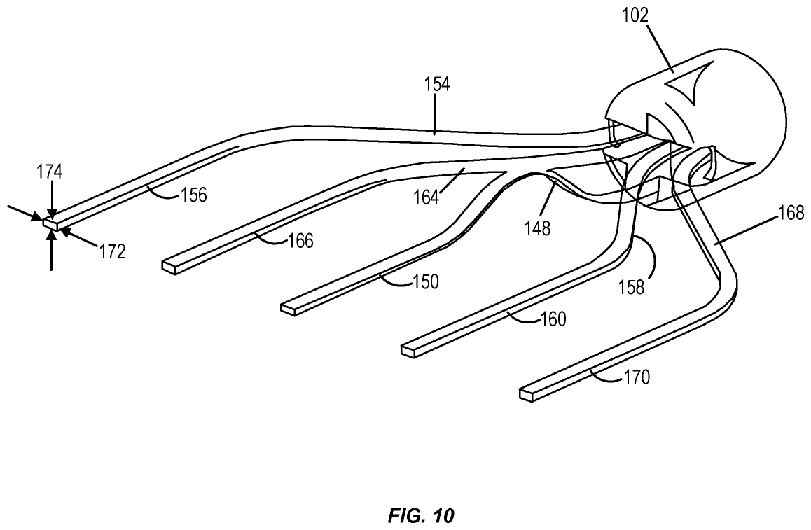

In many embodiments, each of the central spline assembly 104, the first flexible spline frame assembly 106, and the second flexible spline frame assembly 108 includes a flexible understructure member as shown in FIG. 8, FIG. 9, and FIG. 10. The respective flexible understructure members can be formed from any suitable elastically deformable material (e.g., Nitinol). In the illustrated embodiment, the central flexible spline assembly 104 includes a central spline understructure member 146 that is attached to and extends distally from the connector 102. The central spline understructure member 146 has a first proximal portion 148 that is connected to and extends distally from the connector 102 and a first electrode support portion 150 that extends distally from the distal end of the first proximal portion 148. The first proximal portion 148 forms part of the first proximal portion 109 of the central flexible spline assembly 104. The first electrode support portion 150 forms part of the first electrode portion 110 of the central flexible spline assembly 104. The first flexible spline frame assembly 106 includes a first spline frame understructure member 152. The first spline frame understructure member 152 includes a second proximal portion 154, a second electrode portion 156, a third proximal portion 158, and a third electrode portion 160. Each of the second proximal portion 154 and the third proximal portion 158 is attached to and extends distally from the connector 102. The second electrode portion 156 extends distally from the distal end of the second proximal portion 154. The third electrode portion 160 extends distally from the distal end of the third proximal portion 158. The second proximal portion 154 forms part of the second proximal portion 115 of the first flexible spline frame assembly 106. The second electrode portion 156 forms part of the second electrode portion of the first flexible spline frame assembly 106. The third proximal portion 158 forms part of the third proximal portion 119 of the first flexible spline frame assembly 106. The third electrode portion 160 forms part of the third electrode portion 118 of the first flexible spline frame assembly 106. The second flexible spline frame assembly 108 includes a second spline frame understructure member 162. The second spline frame understructure member 162 includes a fourth proximal portion 164, a fourth electrode portion 166, a fifth proximal portion 168, and a fifth electrode portion 170. Each of the fourth proximal portion 164 and the fifth proximal portion 168 is attached to and extends distally from the connector 102. The fourth electrode portion 166 extends distally from the distal end of the fourth proximal portion 164. The fifth electrode portion 170 extends distally from the distal end of the fifth proximal portion 168. The fourth proximal portion 164 forms part of the fourth proximal portion 121 of the second flexible spline frame assembly 108. The fourth electrode portion 166 forms part of the fourth electrode portion 122 of the second flexible spline frame assembly 108. The fifth proximal portion 168 forms part of the fifth proximal portion 125 of the second flexible spline frame assembly 108. The fifth electrode portion 170 forms part of the fifth electrode portion 124 of the second flexible spline frame assembly 108.

As shown in FIG. 9, the first proximal portion 148 of the central spline understructure member 146 has an undulated shape that increases the overall centerline length of the first proximal portion 148 from the proximal end of the first proximal portion 148 to the distal end of the first proximal portion 148 to better match the overall centerline length of each of the proximal portions 154, 158 of the first flexible spline frame assembly 106 and each of the proximal portions 164, 168 of the second flexible spline frame assembly 108. By increasing the overall centerline distance of the first proximal portion 148, the central spline understructure member 146 has an increased bending flexibility compatible with the bending flexibility of each of the first spline frame understructure member 152 and the second spline frame understructure member 162. The undulated shape of the first proximal portion 148 also serves to offset the proximal end of the first proximal portion 148 from the proximal ends of the proximal portions 154, 158, 164, 168, thereby providing for a compact collapsed configuration for introduction through a sheath.

As shown in FIG. 10, which shows a cross-sectional view AA defined in FIG. 9, each of the first proximal portion 148 and the first electrode portion 150; each of the second proximal portion 154, the second electrode portion 156, the third proximal portion 158, and the third electrode portion 160 of the first flexible spline frame understructure member 152: and each of the fourth proximal portion 164, the fourth electrode portion 166, the fifth proximal portion 168, and the fifth electrode portion 170 of the second flexible spline frame substructure member 162 has a rectangular cross-section with a width 172 and a height 174 that is less than the width 172 to bias the flexibility of the distal electrode assembly 101 to enhance the conformability of the distal electrode assembly 101 with a tissue surface.

As shown in FIG. 8, the first spline frame understructure member 152 includes the end portion 120 and the second spline frame understructure member 162 includes the end portion 126. Each of the end portions 120, 126 has a width 176 that is less than the width 172 of the respective electrode portions and proximal portions. The reduced width 176 serves to increase transverse bending flexibility of the end portions 120, 126 to reduce induced strains in the collapsed configurations of the first spline frame understructure member 152 and the second spline frame understructure member 162.

In the illustrated embodiment, each of the proximal portions 148, 154, 158, 164, 168 and the electrode portions 150, 156, 160, 166, 170 of the understructure members 146, 152, 162 is enclosed within a non-conductive shell. The non-conductive shell can include a tube that defines a longitudinally extending lumen in which the respective understructure member is disposed. The non-conductive shell can be formed from any suitable non-conductive material, such as a suitable polymer material.

Among other things, the distal electrode assembly 101 can be configured for use to: (1) define regional propagation maps for tissue surface areas (e.g., one centimeter square areas) of an interior atrial wall of the heart; (2) identify complex fractionated atrial electrograms for ablation; (3) identify localized, focal potentials between the electrodes for higher electrogram resolution; and/or (4) more precisely target areas for ablation. In many embodiments, the distal electrode assembly 101 is configured to be conformable to, and remain in contact with, cardiac tissue even in the presence of erratic cardiac motion, thereby avoiding mapping error(s) and/or ablation problems that can occur as a result of intermittent tissue-electrode contact.

Additionally, the distal electrode assembly 101 may be useful for epicardial and/or endocardial use. For example, the distal electrode assembly 101 may be used in an epicardial procedure where the distal electrode assembly 101 is positioned between the myocardial surface and the pericardium. Alternatively, the distal electrode assembly 101 may be used in an endocardial procedure to quickly sweep and/or analyze the inner surfaces of the myocardium and quickly create high-density maps of the heart tissue's electrical properties.

In many embodiments, the distal electrode assembly 101 includes one or more location sensors 144, such as an electromagnetic location sensor. For example, as illustrated in FIG. 3, the distal portion 112 of the central spline assembly 104 can house a location sensor 144 between the distal end of the first electrode portion 110 and the spline frame support aperture 130. One or more location sensors 144 can be disposed within any suitable portion of the central spline assembly 104 including, but not limited to, distal to the electrodes 114, in between any two of the electrodes 114, and/or proximal to the electrodes 114. The position and orientation of the location sensor(s) 144 within a patient's body can be determined as discussed herein. In a similar manner, one or more location sensors 144 can be disposed within any suitable portion of the first flexible spline frame assembly 106 and/or within any suitable portion of the second flexible spline frame assembly 108, within the connector 102, and/or within the catheter shaft 132. In many embodiments, the high-density catheter 100 includes one or more locations sensors 144 in the catheter shaft 132 for determining and tracking positions and orientations of the catheter shaft 132. In many embodiments, the location sensor(s) 144 is configured to sense a position and orientation of the location sensor(s) 144 with five degrees of freedom (5 DOF). In many embodiments, two location sensors 144 are used to sense a position and orientation of the high-density catheter 100 to six degrees of freedom (6 DOF). As discussed herein, the location sensor(s) 144 can be disposed in a magnetic field and produce one or more signals indicative of the position and orientation of the location sensor(s) 144.

### Localization Systems

The high-density catheter 100 can be used in conjunction with any suitable medical device localization system, such as those referenced and/or described herein. For example, the high-density catheter 100 can be used in conjunction with the catheter localization systems and methods described in U.S. Pat. Pub. No. 2020/0138334 A1 entitled “Method for Medical Device Localization based on Magnetic and Impedance Sensors”, the entire disclosure of which is incorporated herein by reference.

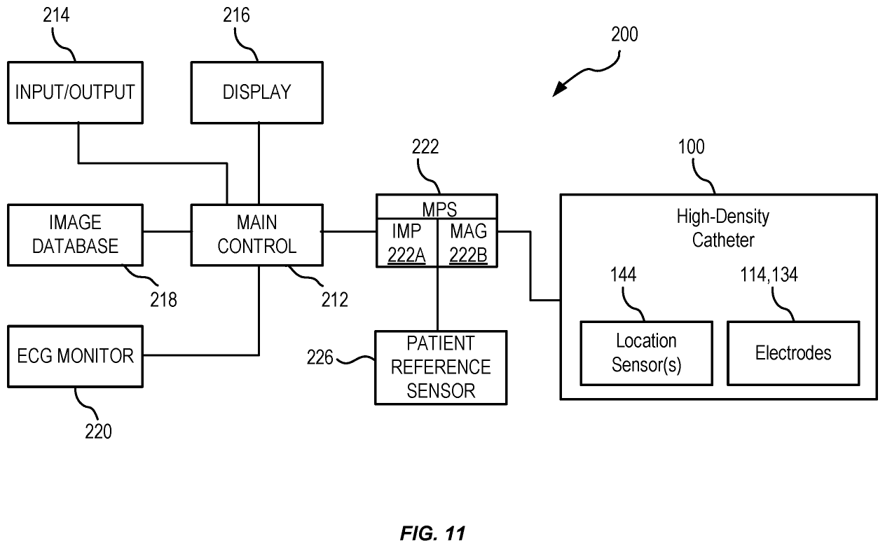

FIG. 11 is a diagrammatic view of a medical device localization system 200 that can be used in conjunction with the high-density catheter 100. The system 200 includes a main electronic control unit 212 (e.g., a processor) having various input/output mechanisms 214, a display 216, an optional image database 218, an electrocardiogram (ECG) monitor 220, a localization system, such as a medical positioning system 222, and the high-density catheter 100. As described herein, in some embodiments the high-density catheter 100 includes the electrodes 114, 134 and one or more of the location sensors 144 (which are in some embodiments configured as magnetic location sensors).

The input/output mechanisms 214 may include conventional apparatus for interfacing with a computer-based control unit including, for example, one or more of a keyboard, a mouse, a tablet, a foot pedal, a switch and/or the like. The display 216 may also comprise conventional apparatus, such as a computer monitor.

Various embodiments described herein may find use in navigation applications that use real-time and/or pre-acquired images of a region of interest. Therefore, the system 200 may optionally include the image database 218 to store image information relating to the patient's body. Image information may include, for example, a region of interest surrounding a destination site for the high-density catheter 100 and/or multiple regions of interest along a navigation path contemplated to be traversed by the high-density catheter 100. The data in the image database 218 may include known image types including (1) one or more two-dimensional still images acquired at respective, individual times in the past; (2) a plurality of related two-dimensional images obtained in real-time from an image acquisition device (e.g., fluoroscopic images from an x-ray imaging apparatus), wherein the image database 218 acts as a buffer (live fluoroscopy); and/or (3) a sequence of related two-dimensional images defining a cine-loop wherein each image in the sequence has at least an ECG timing parameter associated therewith, adequate to allow playback of the sequence in accordance with acquired real-time ECG signals obtained from the ECG monitor 220. It should be understood that the foregoing embodiments are examples only and not limiting in nature. For example, the image database 218 may also include three-dimensional image data as well. It should be further understood that the images may be acquired through any imaging modality, now known or hereafter developed, for example X-ray, ultra-sound, computerized tomography, nuclear magnetic resonance or the like.

The ECG monitor 220 is configured to continuously detect an electrical timing signal of the heart organ through the use of a plurality of ECG electrodes (not shown), which may be externally-affixed to the outside of a patient's body. The timing signal generally corresponds to a particular phase of the cardiac cycle, among other things. Generally, the ECG signal(s) may be used by the control unit 212 for ECG synchronized play-back of a previously captured sequence of images (cine loop) stored in the database 218. The ECG monitor 220 and ECG-electrodes may both include conventional components.

Another medical positioning system sensor, namely, a patient reference sensor (PRS) 226 (if provided in the system 200) can be configured to provide a positional reference of the patient's body so as to allow motion compensation for patient body movements, such as respiration-induced movements. Such motion compensation is described in greater detail in U.S. patent application Ser. No. 12/650,932, entitled “Compensation of Motion in a Moving Organ Using an Internal Position Reference Sensor”, hereby incorporated by reference in its entirety as though fully set forth herein. The PRS 26 may be attached to the patient's manubrium sternum or other location. The PRS 26 can be configured to detect one or more characteristics of the magnetic field in which it is disposed, wherein medical positioning system 222 determines a location reading (e.g., a P&O reading) indicative of the PRS's position and orientation in the magnetic reference coordinate system.

The medical positioning system 222 is configured to serve as the localization system and therefore to determine position (localization) data with respect to the one or more location sensors 144 and/or the electrodes 114, 134 and output a respective location reading. In an embodiment, the medical positioning system 222 may include a first medical positioning system or an electrical impedance-based medical positioning system 222A that determines locations of the electrodes 114, 134 in a first coordinate system, and a second medical positioning system or magnetic field-based medical positioning system 222B that determines location(s) of the location sensor(s) 144 in a second coordinate system. In an embodiment, the location readings may each include at least one or both of a position and an orientation (P&O) relative to a reference coordinate system (e.g., magnetic based coordinate system or impedance based coordinate system). For some types of sensors, the P&O may be expressed with five degrees-of-freedom (five DOF) as a three-dimensional (3D) position (e.g., a coordinate in three perpendicular axes X, Y and Z) and two-dimensional (2D) orientation (e.g., a pitch and yaw) of the location sensor(s) 144 in a magnetic field relative to a magnetic field generator(s) or transmitter(s) and/or the electrodes 114, 134 in an applied electrical field relative to an electrical field generator (e.g., a set of electrode patches). For other sensor types, the P&O may be expressed with six degrees-of-freedom (six DOF) as a 3D position (e.g., X, Y, Z coordinates) and 3D orientation (e.g., roll, pitch, and yaw).

The impedance based medical positioning system 222A determines locations of the electrodes 114, 134 based on capturing and processing signals received from the electrodes 114, 134 and external electrode patches while the electrodes 114, 134 are disposed in a controlled electrical field (e.g., potential field) generated by the electrode patches, for example. FIG. 12 is a diagrammatic overview of an exemplary embodiment of the electrical impedance-based medical positioning system (‘MPS system’) 222A. The MPS system 222A may include various visualization, mapping and navigation components as known in the art. including, for example, an EnSite™ X EP System commercially available from Abbott Laboratories or as seen generally by reference to U.S. Pat. No. 7,263,397 entitled “Method and Apparatus for Catheter Navigation and Location and Mapping in the Heart” to Hauck et al., or U.S. Patent Publication No. 2007/0060833 A1 to Hauck entitled “Method of Scaling Navigation Signals to Account for Impedance Drift in Tissue”, both owned by the common assignee of the present invention, and both hereby incorporated by reference in their entireties.

The magnetic-based medical positioning system 222B determines locations (e.g., P&O) of the location sensor(s) 144 in a magnetic coordinate system based on capturing and processing signals received from the location sensor(s) 144 while the location sensor 144 is disposed in a controlled low-strength alternating current (AC) magnetic (e.g., magnetic) field. Each location sensor 144 and the like may include a coil and, from an electromagnetic perspective, the changing or AC magnetic field may induce a current in the coil(s) when the coil(s) are in the magnetic field. The location sensor(s) 144 is thus configured to detect one or more characteristics (e.g., flux) of the magnetic field(s) in which it is disposed and generate a signal indicative of those characteristics, which is further processed by medical positioning system 222B to obtain a respective P&O for the location sensor(s) 144 relative to, for example, a magnetic field generator.

It should be understood that the high-density catheter 100 may be used for any other suitable diagnostic and/or therapeutic purposes. Accordingly, the high-density catheter 100 can be configured to perform ablation procedures, cardiac mapping, electrophysiological (EP) studies and other diagnostic and/or therapeutic procedures. Embodiments are not limited to any one type of catheter or catheter-based system or procedure.

### Applications

The high-density catheter 100 can be used in conjunction with any suitable catheter system, such as those referenced and/or described herein. For example, the high-density catheter 100 can be used to generate an electrophysiological map of electrical activity within a patient's heart to diagnose cardiac arrythmias. The high-density catheter 100 can be used to selectively alter the patient's heart tissue to reduce or eliminate the pathological electrical condition to reduce or eliminate occurrence of the cardiac arrythmia. The high-density catheter 100 can configured for use in performing any suitable treatment, such as, but not limited to, radio frequency (RF) ablation, pulsed field ablation (PFA), cryoablation, laser ablation, chemical ablation, high-intensity focused ultrasound ablation, microwave ablation, and/or other ablation treatments.

For example, and in some embodiments, the high-density catheter 100 may be configured as a bipolar electrode assembly for use in bipolar-based electroporation therapy. Specifically, the electrodes 114, 134 of the high-density catheter 100 can be individually electrically coupled to an electroporation generator (e.g., via suitable electrical wire or other suitable electrical conductors extending through the catheter shaft 132) and are configured to be selectively energized by the electroporation generator 26 with opposite polarities to generate a potential and corresponding electric field therebetween, for PFA therapy. That is, one of electrodes 114, 134 can be configured to function as a cathode, and another of the electrodes 114, 134 can be configured to function as an anode. Any suitable combination of the electrodes 114 of the electrode assembly 101 can be used as anodes and cathodes. For example, all the electrodes 114 on one of the electrode portions 110, 116, 118, 122, 124 can be employed as a cathode and all the electrodes 114 on an adjacent one of the electrode portions 110, 116, 118, 122, 124 can be employed as an anode. As another example, every other of the electrodes 114 along one of the electrode portions 110, 116, 118, 122, 124 can be employed as a cathode and the other of the electrodes 114 along the electrode portion can be employed as an anode. The electrodes 114, 134 may be any suitable electroporation electrodes. In the exemplary embodiment, the electrodes 114, 134 are ring electrodes. The electrodes 114, 134, however, may have any other suitable shape or configuration. It is realized that the shape, size, and/or configuration of the electrodes 114, 134 may impact various parameters of the applied electroporation therapy. For example, increasing the surface area of one or both of the electrodes 114, 134 may reduce the applied voltage needed to cause the same level of tissue destruction. Moreover, although each of the electrodes 114, 134 is illustrated as a single electrode, either or both of the electrodes 114 and the electrodes 134 may be alternatively embodied as two or more discrete electrodes.

Ablation therapy may be used to treat various conditions afflicting the human anatomy. One such condition in which ablation therapy may be used is the treatment of cardiac arrhythmias. When tissue is ablated, or at least subjected to ablative energy generated by an ablation generator and delivered by an ablation catheter, lesions form in the tissue. Electrodes mounted on or in ablation catheters are used to create tissue necrosis in cardiac tissue to correct conditions such as atrial arrhythmia (including, but not limited to, ectopic atrial tachycardia, atrial fibrillation, and atrial flutter). Arrhythmias can create a variety of dangerous conditions including loss of synchronous atrioventricular contractions and stasis of blood flow. It is believed that the primary cause of atrial arrhythmia is stray electrical signals within the left or right atrium of the heart. The ablation catheter imparts ablative energy (e.g., radiofrequency energy, PFA, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric fields that induce pore formation in the cellular membrane. The electric field may be induced by applying a relatively short duration pulse which may last, for example, from a nanosecond to several milliseconds. Such a pulse may be repeated to form a pulse train. When such an electric field is applied to tissue in an in vivo setting, the cells in the tissue are subjected to a trans-membrane potential, which opens the pores on the cell wall. Electroporation may be reversible (i.e., the temporally-opened pores will reseal) or irreversible (i.e., the pores will remain open), causing cellular destruction. For example, in the field of gene therapy, reversible electroporation is used to transfect high molecular weight therapeutic vectors into the cells. In other therapeutic applications, a suitably configured pulse train alone may be used to cause cell destruction, for instance by causing irreversible electroporation.

In some embodiments, the high-density catheter 100 is used for electroporation-induced primary necrosis therapy, which refers to the effects of delivering electrical current in such manner as to directly cause an irreversible loss of plasma membrane (cell wall) integrity leading to its breakdown and cell necrosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field (i.e., pulsed field ablation (PFA)) in the form of short-duration pulses (e.g., 0.1 to 20 ms duration) between closely spaced electrodes capable of delivering an electric field strength of about 0.1 to 1.0 kV/cm.

In one or more embodiments of the present disclosure, a catheter includes an elongated catheter shaft and an electrode assembly. The electrode assembly includes a proximal connector, a central spline assembly, a first frame assembly, and a second frame assembly. The proximal connector is attached to a distal end of the elongated catheter shaft. The central spline assembly includes a first proximal portion, a first electrode portion, a first spline distal portion, and first spline electrodes. The first proximal portion is attached to and extends distally from the proximal connector. The first electrode portion extends distally from the first proximal portion. The first spline distal portion extends distally from a distal end of the first electrode portion. The first spline distal portion includes a spline frame support aperture. The first spline electrodes are distributed along the first electrode portion. The first frame assembly includes a second proximal portion, a second electrode portion, a third proximal portion, a third electrode portion, a first frame end portion, second spline electrodes, and third spline electrodes. The second proximal portion is attached to and extends distally from the proximal connector on a first side of the central spline assembly. The second electrode portion extends distally from the second proximal portion. The third proximal portion is attached to and extends distally from the proximal connector on a second side of the central spline assembly opposite to the first side of the central spline assembly. The third electrode portion extends distally from the third proximal portion. The first frame end portion extends between a distal end of each of the second electrode portion and the third electrode portion. The first frame end portion extends through the spline frame support aperture. The second spline electrodes are distributed along the second electrode portion. The third spline electrodes are distributed along the third electrode portion. The second frame assembly includes a fourth proximal portion, a fourth electrode portion, a fifth proximal portion, a fifth electrode portion, a second frame end portion, fourth spline electrodes, and fifth spline electrodes. The fourth proximal portion is attached to and extends distally from the proximal connector on the first side of the central spline assembly. The fourth electrode portion extends distally from the fourth proximal portion. The fifth proximal portion is attached to and extends distally from the proximal connector on the second side of the central spline assembly. The fifth electrode portion extends distally from the fifth proximal portion. The second frame end portion extends between a distal end of each of the fourth electrode portion and the fifth electrode portion. The second frame end portion extends through the spline frame support aperture. The fourth spline electrodes are distributed along the fourth electrode portion. The fifth spline electrodes are distributed along the fifth electrode portion. Optionally, the first electrode portion, the second electrode portion, the third electrode portion, the fourth electrode portion, and the fifth electrode portion are coplanar in an expanded configuration of the electrode assembly. Optionally, the third electrode portion is disposed between the first electrode portion and the fifth electrode portion in the expanded configuration of the electrode assembly and the fourth electrode portion is disposed between the first electrode portion and the second electrode portion in the expanded configuration of the electrode assembly. Optionally, the electrode assembly is configured to expand from a collapsed configuration to an expanded configuration when introduced through a sheath. Optionally, the first frame end portion extends distally from the distal end of each of the second electrode portion and the third electrode portion to a distal most point of the first frame end portion in the expanded configuration; the distal most point of the first frame end portion remains on the first side of the central spline assembly and moves distally relative to the central spline assembly during contraction of the electrode assembly from the expanded configuration to the collapsed configuration; the second frame end portion extends distally from the distal end of each of the fourth electrode portion and the fifth electrode portion to a distal most point of the second frame end portion in the expanded configuration; and the distal most point of the second frame end portion remains on the second side of the central spline assembly and moves distally relative to the central spline assembly during contraction of the electrode assembly from the expanded configuration to the collapsed configuration. Optionally, a respective segment of each of the first frame end portion and the second frame end portion moves through the spline frame support aperture during reconfiguration of the electrode assembly from the collapsed configuration to the expanded configuration. Optionally, a single configuration is used for both the first frame assembly and the second frame assembly; and an orientation of the second frame assembly in the electrode assembly is rotated 180 degrees relative to the orientation of the first frame assembly in the electrode assembly. Optionally, the first frame end portion crosses the second frame end portion within the spline frame support aperture. Optionally, the central spline assembly includes a central spline magnetic position sensor. Optionally, the central spline assembly includes a proximal portion with an undulated shape disposed between the first spline electrodes and the proximal connector. Optionally, the first frame assembly includes a first frame magnetic position sensor. Optionally, the second frame assembly includes a second frame magnetic position sensor. Optionally, the electrode assembly is configured to conform to a tissue to place the first spline electrodes, the second spline electrodes, the third spline electrodes, the fourth spline electrodes, and the fifth spline electrodes in contact with the tissue. Optionally, a center-to-center distance between adjacent electrodes of the first spline electrodes, the second spline electrodes, the third spline electrodes, the fourth spline electrodes, and the fifth spline electrodes is between 2 and 10 millimeters. Optionally, each of the first spline electrodes, the second spline electrodes, the third spline electrodes, the fourth spline electrodes, and the fifth spline electrodes includes at least 4 electrodes. Optionally, the first spline electrodes, the second spline electrodes, the third spline electrodes, the fourth spline electrodes, and the fifth spline electrodes are arranged in a rectangular planar area. Optionally, the catheter further includes a fluid delivery lumen fluidly coupled to an irrigation fluid source and configured to deliver an irrigation fluid to the electrode assembly. Optionally, the electrode assembly is configured for ablation therapy.

In one or more embodiments of the present disclosure, a catheter system includes any one of the catheter configurations described herein and controller circuitry. The controller circuitry is communicatively coupled to the first spline electrodes, the second spline electrodes, the third spline electrodes, the fourth spline electrodes, and the fifth spline electrodes. The controller circuitry is configured to sample electrical signals received from the first spline electrodes, the second spline electrodes, the third spline electrodes, the fourth spline electrodes, and the fifth spline electrodes. Optionally, the catheter system further includes a display communicatively coupled to the controller circuitry. Optionally, the controller circuitry is configured to generate and display a map on the display indicative of one or more electrical characteristics of tissue contacted by the first spline electrodes, the second spline electrodes, the third spline electrodes, the fourth spline electrodes, and the fifth spline electrodes.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having.” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A catheter comprising:
an elongated catheter shaft; and
an electrode assembly comprising:
a proximal connector attached to a distal end of the elongated catheter shaft;
a central spline assembly comprising a first proximal portion, a first electrode portion, a first spline distal portion, and first spline electrodes, wherein the first proximal portion is attached to and extends distally from the proximal connector, wherein the first electrode portion extends distally from the first proximal portion, wherein the first spline distal portion extends distally from a distal end of the first electrode portion and comprises a spline frame support aperture, and wherein the first spline electrodes are distributed along the first electrode portion;
a first frame assembly comprising a second proximal portion, a second electrode portion, a third proximal portion, a third electrode portion, a first frame end portion, second spline electrodes, and third spline electrodes, wherein the second proximal portion is attached to and extends distally from the proximal connector on a first side of the central spline assembly, wherein the second electrode portion extends distally from the second proximal portion, wherein the third proximal portion is attached to and extends distally from the proximal connector on a second side of the central spline assembly opposite to the first side of the central spline assembly, wherein the third electrode portion extends distally from the third proximal portion, wherein first frame end portion extends between a distal end of each of the second electrode portion and the third electrode portion and extends through the spline frame support aperture, wherein the second spline electrodes are distributed along the second electrode portion, and wherein the third spline electrodes are distributed along the third electrode portion; and
a second frame assembly comprising a fourth proximal portion, a fourth electrode portion, a fifth proximal portion, a fifth electrode portion, a second frame end portion, fourth spline electrodes, and fifth spline electrodes, wherein the fourth proximal portion is attached to and extends distally from the proximal connector on the first side of the central spline assembly, wherein the fourth electrode portion extends distally from the fourth proximal portion, wherein the fifth proximal portion is attached to and extends distally from the proximal connector on the second side of the central spline assembly, wherein the fifth electrode portion extends distally from the fifth proximal portion, wherein the second frame end portion extends between a distal end of each of the fourth electrode portion and the fifth electrode portion and extends through the spline frame support aperture, wherein the fourth spline electrodes are distributed along the fourth electrode portion, and wherein the fifth spline electrodes are distributed along the fifth electrode portion.

2. The catheter of claim 1, wherein the first electrode portion, the second electrode portion, the third electrode portion, the fourth electrode portion, and the fifth electrode portion are coplanar in an expanded configuration of the electrode assembly.

3. The catheter of claim 2, wherein:
the third electrode portion is disposed between the first electrode portion and the fifth electrode portion in the expanded configuration of the electrode assembly; and
the fourth electrode portion is disposed between the first electrode portion and the second electrode portion in the expanded configuration of the electrode assembly.

4. The catheter of claim 1, wherein the electrode assembly is configured to expand from a collapsed configuration to an expanded configuration when introduced through a sheath.

5. The catheter of claim 4, wherein:
the first frame end portion extends distally from the distal end of each of the second electrode portion and the third electrode portion to a distal most point of the first frame end portion in the expanded configuration;
the distal most point of the first frame end portion remains on the first side of the central spline assembly and moves distally relative to the central spline assembly during contraction of the electrode assembly from the expanded configuration to the collapsed configuration;
the second frame end portion extends distally from the distal end of each of the fourth electrode portion and the fifth electrode portion to a distal most point of the second frame end portion in the expanded configuration; and
the distal most point of the second frame end portion remains on the second side of the central spline assembly and moves distally relative to the central spline assembly during contraction of the electrode assembly from the expanded configuration to the collapsed configuration.

6. The catheter of claim 4, wherein a respective segment of each of the first frame end portion and the second frame end portion moves through the spline frame support aperture during reconfiguration of the electrode assembly from the collapsed configuration to the expanded configuration.

7. The catheter of claim 1, wherein:
a single configuration is used for both the first frame assembly and the second frame assembly; and
an orientation of the second frame assembly in the electrode assembly is rotated 180 degrees relative to the orientation of the first frame assembly in the electrode assembly.

8. The catheter of claim 1, wherein the first frame end portion crosses the second frame end portion within the spline frame support aperture.

9. The catheter of claim 1, wherein the central spline assembly comprises a central spline magnetic position sensor.

10. The catheter of claim 1, wherein the central spline assembly comprises a proximal portion with an undulated shape disposed between the first spline electrodes and the proximal connector.

11. The catheter of claim 1, wherein the first frame assembly comprises a first frame magnetic position sensor.

12. The catheter of claim 11, wherein the second frame assembly comprises a second frame magnetic position sensor.

13. The catheter of claim 1, wherein the electrode assembly is configured to conform to a tissue to place the first spline electrodes, the second spline electrodes, the third spline electrodes, the fourth spline electrodes, and the fifth spline electrodes in contact with the tissue.

14. The catheter of claim 1, wherein a center-to-center distance between adjacent electrodes of the first spline electrodes, the second spline electrodes, the third spline electrodes, the fourth spline electrodes, and the fifth spline electrodes is between 2 and 10 millimeters.

15. The catheter of claim 1, wherein each of the first spline electrodes, the second spline electrodes, the third spline electrodes, the fourth spline electrodes, and the fifth spline electrodes comprises at least 4 electrodes.

16. The catheter of claim 1, wherein the first spline electrodes, the second spline electrodes, the third spline electrodes, the fourth spline electrodes, and the fifth spline electrodes are arranged in a rectangular planar area.

17. The catheter of claim 1, further comprising a fluid delivery lumen fluidly coupled to an irrigation fluid source and configured to deliver an irrigation fluid to the electrode assembly.

18. The catheter of claim 1, wherein the electrode assembly is configured for ablation therapy.

19. A catheter system comprising:
a catheter comprising an elongated catheter shaft and an electrode assembly comprising:
a proximal connector attached to a distal end of the elongated catheter shaft;
a central spline assembly comprising a first proximal portion, a first electrode portion, a first spline distal portion, and first spline electrodes, wherein the first proximal portion is attached to and extends distally from the proximal connector, wherein the first electrode portion extends distally from the first proximal portion, wherein the first spline distal portion extends distally from a distal end of the first electrode portion and comprises a spline frame support aperture, and wherein the first spline electrodes are distributed along the first electrode portion;
a first frame assembly comprising a second proximal portion, a second electrode portion, a third proximal portion, a third electrode portion, a first frame end portion, second spline electrodes, and third spline electrodes, wherein the second proximal portion is attached to and extends distally from the proximal connector on a first side of the central spline assembly, wherein the second electrode portion extends distally from the second proximal portion, wherein the third proximal portion is attached to and extends distally from the proximal connector on a second side of the central spline assembly opposite to the first side of the central spline assembly, wherein the third electrode portion extends distally from the third proximal portion, wherein first frame end portion extends between a distal end of each of the second electrode portion and the third electrode portion and extends through the spline frame support aperture, wherein the second spline electrodes are distributed along the second electrode portion, and wherein the third spline electrodes are distributed along the third electrode portion; and
a second frame assembly comprising a fourth proximal portion, a fourth electrode portion, a fifth proximal portion, a fifth electrode portion, a second frame end portion, fourth spline electrodes, and fifth spline electrodes, wherein the fourth proximal portion is attached to and extends distally from the proximal connector on the first side of the central spline assembly, wherein the fourth electrode portion extends distally from the fourth proximal portion, wherein the fifth proximal portion is attached to and extends distally from the proximal connector on the second side of the central spline assembly, wherein the fifth electrode portion extends distally from the fifth proximal portion, wherein the second frame end portion extends between a distal end of each of the fourth electrode portion and the fifth electrode portion and extends through the spline frame support aperture, wherein the fourth spline electrodes are distributed along the fourth electrode portion, and wherein the fifth spline electrodes are distributed along the fifth electrode portion; and
controller circuitry communicatively coupled to the first spline electrodes, the second spline electrodes, the third spline electrodes, the fourth spline electrodes, and the fifth spline electrodes, and configured to sample electrical signals received from the first spline electrodes, the second spline electrodes, the third spline electrodes, the fourth spline electrodes, and the fifth spline electrodes.

20. The catheter system of claim 19, further comprising a display communicatively coupled to the controller circuitry, wherein the controller circuitry is configured to generate and display a map indicative of one or more electrical characteristics of tissue contacted by the first spline electrodes, the second spline electrodes, the third spline electrodes, the fourth spline electrodes, and the fifth spline electrodes.

21-37. (canceled)
